(19) United States
(12) Patent Application Publication
(10) Pub. No.: US 2006/0030871 A1
(43) Pub. Date: Feb. 9, 2006

(54) Vascular tunneler

(76) Inventors: Matthew Hain, Wayne, NJ (US); Jamie Henderson, Oakland, NJ (US)

Correspondence Address: RATNERPRESTIA, P O BOX 980, VALLEY FORGE, PA, 19482-0980, US

(21) Appl. No.: 10/913,266

(22) Filed: Aug. 5, 2004

(51) Int. Cl. A61B 17/00

(52) U.S. Cl. 606/190

(57) ABSTRACT

The present invention is a tunneling device for vascular tunneling procedures. The invention includes a tunneling device and a tunneling tip. The tunneling device includes a tissue-separating source of energy at the tunneling tip. The preferred source of energy is ultrasonic movement at the distal tip of the tunneler. Preferably the ultrasonic driver is disposed in a removably attached tip but it may be in the tunneler handle. Also included is a method of tunneling which includes separating tissue layers as the tunneler is advanced. The preferred method uses an ultrasonically vibrating tip.

### FIELD OF THE INVENTION

The present invention generally relates to devices and methods of implanting vascular grafts, and more specifically to tunneling devices for the implantation of vascular grafts.

### BACKGROUND OF THE INVENTION

A variety of methods are known to repair body lumens, including blood vessels such as arteries or veins that have become occluded or stenosed. Typically these methods involve the placement of a vascular graft that is suitable for implantation in the body to reestablish or redirect the flow of blood through or around the affected area. Peripheral vascular graft implantation requires the creation of a subcutaneous pathway commonly called a graft tunnel. Tunneling is a surgical step in vascular procedures but often results in injury to surrounding tissue. This injury is caused by dissection of the tissue and frictional forces on the tissue as the tunnel is created, as well as frictional forces exerted on the tunnel wall by the repair device (e.g., a graft) during movement to, and delivery at, the affected site in need of repair. The degree of this injury has an impact on the healing of the patient.

The conventional approach to creating a graft tunnel is with a device called a graft tunneler. Generally, there are two types of tunnelers: standard tunnelers, and sheath tunnelers. Standard tunnelers draw a vascular graft through a dissected tissue tunnel which is created by insertion of a rigid, bullet tipped rod through a skin incision. One such example uses a two-part tunneler instrument which includes an oversized, relatively rigid metal or plastic hollow tube with a removable bullet shaped dissection tip on one end, and an internal smaller diameter indwelling rod for attaching the vascular graft material.

An example of a sheath tunneler is the Gore tunneler which is produced by W. L. Gore and Associates, Inc. of Flagstaff, Ariz. This two-part tunneler is used to implant a vascular graft subcutaneously with an oversized tissue passageway. The Gore tunneler is comprised of a hollow rigid metal shaft connected to a handle with a removable bullet tip at one end of the shaft. The shaft is fabricated from stainless steel and fits into a formed handle with a center rod. The instrument is used to bluntly dissect a tunnel by forcing the bullet-tipped hollow shaft through the tissue. After suture attachment of the graft material to the inner rod, the vascular graft is then easily drawn back through the entire length of the oversized hollow tube. With the graft positioned in place, but still within the hollow shaft, the hollow shaft is then extracted from the tissue tunnel without extracting the graft from the subcutaneous passageway.

It would therefore be desirable to have an implantable vascular graft that can be implanted with less tissue trauma than that which is caused by tunnelers of the prior art.

### SUMMARY OF THE INVENTION

The present invention includes a tunneling instrument having a tip which has means for delivering tissue-separating energy to tissue cells contacting the tip during use. A preferred tunneling instrument in accordance with the invention has an ultrasonically driven tip that vibrates ultrasonically during use. The preferred device has an ultrasonic horn disposed in the tip of the tunneler and a stack disposed in the shaft. The primary purpose of driving the tip ultrasonically is to reduce the force exerted by the surgeon to create the tunnel in the patient. Reduced tunneling force results in less tissue trauma to the patient which will lead to reduced swelling and shorter recovery times. In addition, the surgeon using less tunneling force will be less likely to injure the patient by mistakenly misguiding the tunneler tip and puncturing an organ which could cause injury or death.

In one embodiment, the tunneler tip is removably connected to the tunneler, preferably by a threaded connection. The removable tip in this embodiment houses the ultrasonic driver which is connected through the tunneler by a power line to a power supply. Another embodiment includes an ultrasonic driver within the tunneler handle.

The present invention also includes just a tunneler tip for use in a surgical tunneling instrument. The tunneler tip comprises a body having a distal end, a proximal end, and a cavity disposed therebetween. Within the cavity is a means for delivering cell tissue separating energy to body tissue. The preferred means include ultrasonic drivers to deliver ultrasonic, vibrational energy to the tip, and monopolar or bipolar tips to deliver electricity directly to the distal tip.

Also included in the present invention is a method of forming a tunnel in living tissue using the devices of the present invention. Specifically, the method comprises the steps of advancing a tunneling device into living tissue and separating tissue layers at the tip of the tunneling device as the tunneling device is advanced through the tissue. The preferred method uses an ultrasonic tunneling device to bluntly dissect tissue between the subcutaneous tissue and fascia tissue layers to form a tunnel therein.

### BRIEF DESCRIPTION OF THE DRAWINGS

FIG. 1 is partial cross-sectional view of an ultrasonic driver in accordance with the present invention;

FIG. 2 illustrates the ultrasonic driver of FIG. 1 with a tip disposed on the distal end of the driver head;

FIG. 3 illustrates an ultrasonic driver in accordance with the present invention with a tip integrally formed with the driver head;

FIG. 4 illustrates the ultrasonic driver of FIG. 2 disposed on the distal end of a tunneler;

FIG. 5 illustrates a variation of that shown in FIG. 4 where the tunneler extends further up the ultrasonic driver;

FIG. 6 illustrates a tunneler of the present invention attached to a power supply;

FIG. 7 illustrates an ultrasonic tunneler tip threadedly connected to a tunneler in accordance with the present invention;

FIG. 8 illustrates an alternative embodiment of the tunneler in accordance with the present invention;

FIG. 9 illustrates the tip of a tunneler in accordance with the present invention having a bipolar tip at its distal end;

FIG. 10 illustrates a bipolar tunneler tip threadedly connected to a tunneler in accordance with the present invention; and

FIG. 11 illustrates an embodiment of the present invention in which an ultrasonic driver is disposed within the tunneler handle.

### DETAILED DESCRIPTION OF THE INVENTION

The use of ultrasonic movement in scalpels and other knives, as well as the use of electrical energy in surgical pencils and the like, is known. The present invention, however, uses one or both of these sources of energy right at the tip of a tunneling device to reduce trauma to body tissue during tunneling procedures or other blunt dissections such as are performed for vascular graft placement. The energy delivered at the tunneler tip in accordance with the present invention also can help cauterize small bleeding vessels during the tunneling procedure. The energy delivered at the tip of the tunneler in accordance with the present invention also eliminates, or at least greatly reduces, the aggressive tunneling force that is applied by the operator as compared to conventional tunneling devices. This delivery of energy (either ultrasonic or electrical) to the tip generally facilitates tissue separation directly in front of the tunneler tip as the tip is advanced through the tissue during tunneling. Moreover, the cells proximate the tunneler tip are influenced by the tip of the tunneler as the tunneler is advanced. By “cells proximate the tunneler tip,” it is meant those cells contacting the tunneler tip, or which are sufficiently near the tunneler tip so as to be affected by the energy delivered through the tunneler tip. Generally, the present invention allows easier tunneling and reduces tissue trauma, recovery time, and pain for the patient.

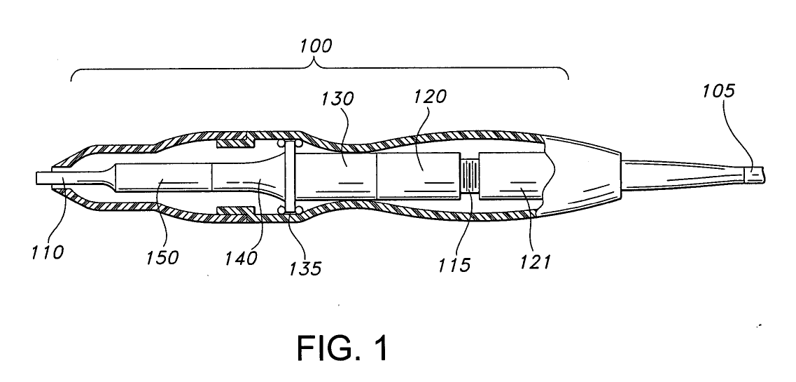

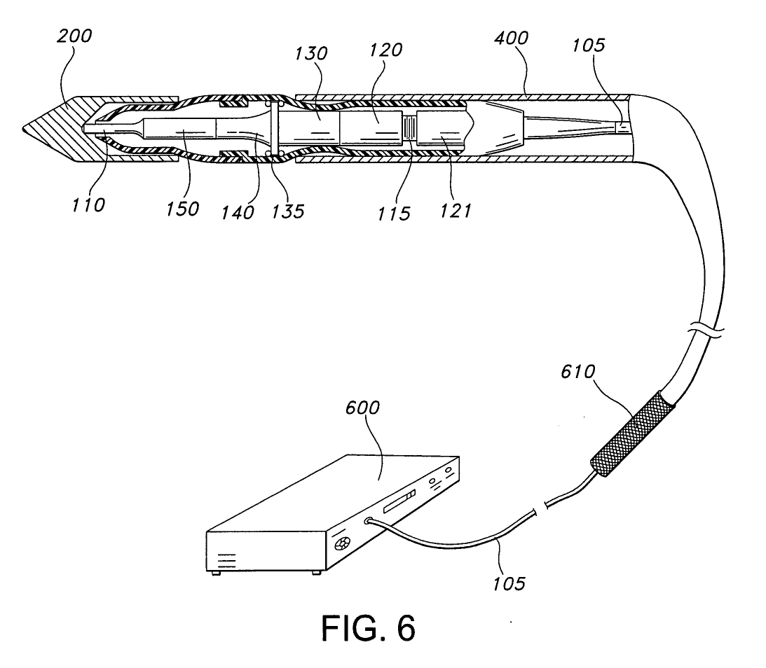

The ultrasonic drivers which can be used in accordance with the present invention are known to those skilled in the art of ultrasonic drivers. By way of example, however, FIG. 1 shows one embodiment of the present invention with an exemplary ultrasonic driver. By means of power line 105, electrical energy, i.e., drive current, is sent from a power supply proximate the tip (shown for example, in FIG. 6, as power supply 600 and discussed in more detail below) to driver 100 where it imparts ultrasonic longitudinal movement to head 110 at the distal end of the device. When power is applied to ultrasonic driver 100, the assembly (discussed in more detail below) will cause head 110 to vibrate longitudinally (for example at approximately 40 kHz). The amount of longitudinal movement will vary proportionately with the amount of driving power (current) applied, as adjustably selected by the user.

Such ultrasonic vibration of the head will generate heat as the head contacts tissue, i.e., the movement of the head through the tissue converts the mechanical energy of the moving head to thermal energy in a very localized area at the tip of the head (and therefore tunneler tip). This localized heat creates a narrow zone of coagulation, which will reduce or eliminate bleeding in small vessels, such as those less than one millimeter in diameter. The degree of hemostasis will vary with the level of driving power applied, the tunneling force applied by the surgeon, the nature of the tissue type, and the vascularity of the tissue, among other factors. Ultrasonic vibration at the tunneler tip will also reduce friction which will result in tunneling with less force exerted by the surgeon.

As illustrated in FIG. 1, this example of a suitable ultrasonic driver 100 houses a piezoelectric transducer 115 for converting electrical energy to mechanical energy that results in longitudinal vibrational motion of the ends of the transducer. Transducer 115 in this embodiment is in the form of a stack of ceramic piezoelectric elements with a motion null point located at some point along the stack, in accordance with the prior art. The transducer stack is mounted between two cylinders 120 and 121. Cylinder 130 is attached to cylinder 120, which in turn is mounted to the housing at another motion null point 135. Horn 140 is also attached to null point 135 on is proximal side and to head 110 coupler 150 on its distal side. Head 110 is affixed to coupler 150. As a result, head 110 will vibrate in the longitudinal direction at an ultrasonic frequency rate with transducer 115.

The parts of the hand piece are designed such that the combination will oscillate at the same resonant frequency. In particular, the elements are preferably tuned such that the resulting length of each such element is one-half wavelength. Longitudinal back and forth motion is amplified as the diameter closer to head 110 of the acoustical mounting horn 140 decreases. Thus, horn 140 as well as coupler 150 are shaped and dimensioned so as to amplify head 110 motion and provide harmonic vibration in resonance with the rest of the acoustic system, which produces the maximum back and forth motion of the end of the acoustical mounting horn 140 close to head 110.

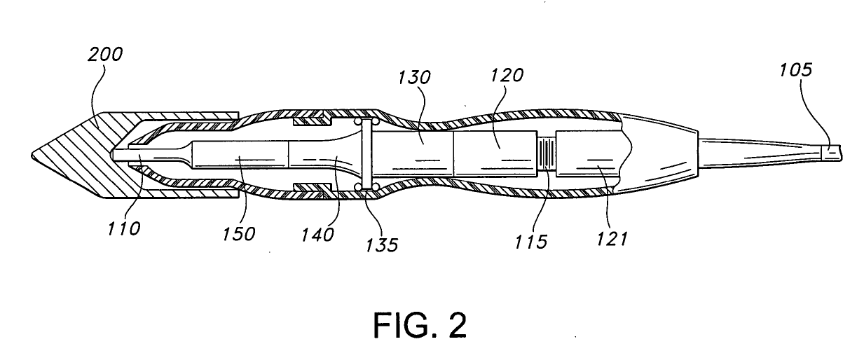

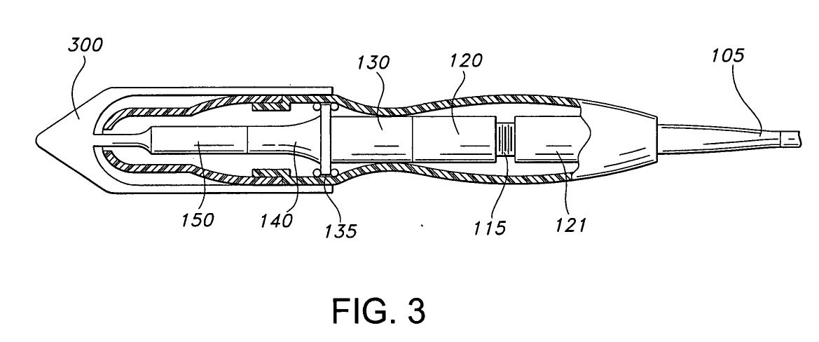

FIG. 2 shows an alternative embodiment of the present invention in which head 110 is covered by tunneler tip 200. In this embodiment, tunneler tip 200 is driven by head 110 and conveys the ultrasonic energy described above to a larger tip to aid in tunneling through tissue. In yet another embodiment, head 110 and tunneler tip 200 could be formed of a single integral piece, such as is shown in FIG. 3 with integral head 300. In either event, head 110 and tunneler tip 200 (or simply head 300) are attached to driver 100 through means known to those skilled in the art.

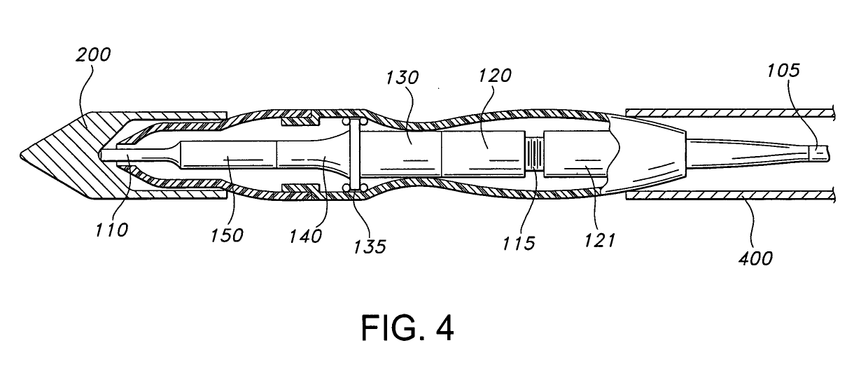

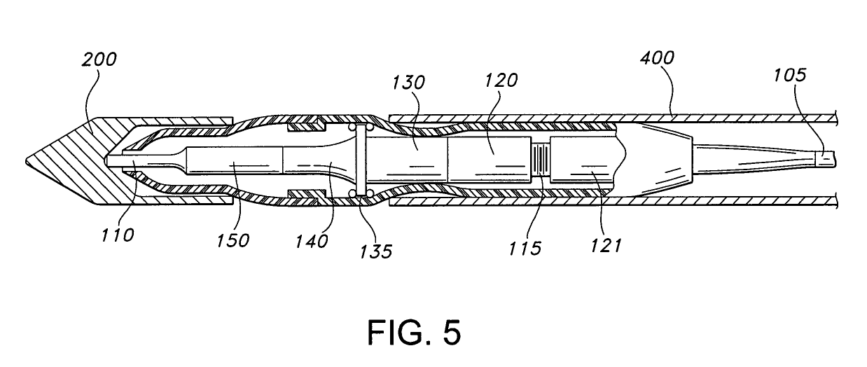

FIG. 4 shows the device of FIG. 2 attached to the end of tunneler 400. The tunnelers used in accordance with the present invention are known to those skilled in the art. The tunnelers may be connected to the tips by known means, including threaded connections. FIG. 5 shows an alternative embodiment where tunneler 400 extends further along driver 100. In yet another embodiment (not shown), the tunneler could extend even further along driver 100 and meet the tip 200 such that the entire driver 100 is contained within the tunneler except for that part covered by tip 200.

FIG. 6 illustrates the driver of FIG. 5 connected to a power supply 600. Power supply 600 is consistent with ultrasonic driver power sources known to those skilled in the art. Power supply 600 provides controllable current to power line 105. Included is handle 610 for the user to grasp and control the tunneler during operation.

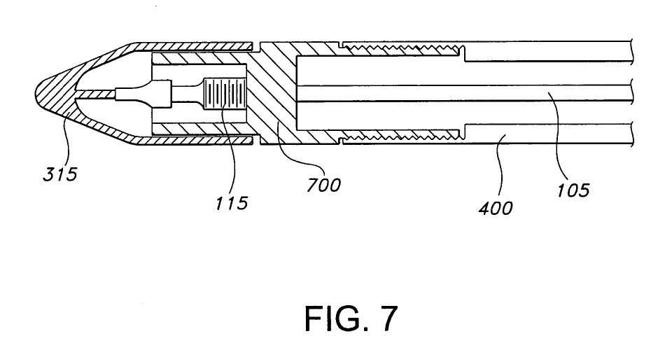

FIG. 7 shows an embodiment of the invention where tunneler 400 is threadedly connected to driver 700. As described above, power line 105 feeds current to transducer 115 which oscillates and drives tip 315.

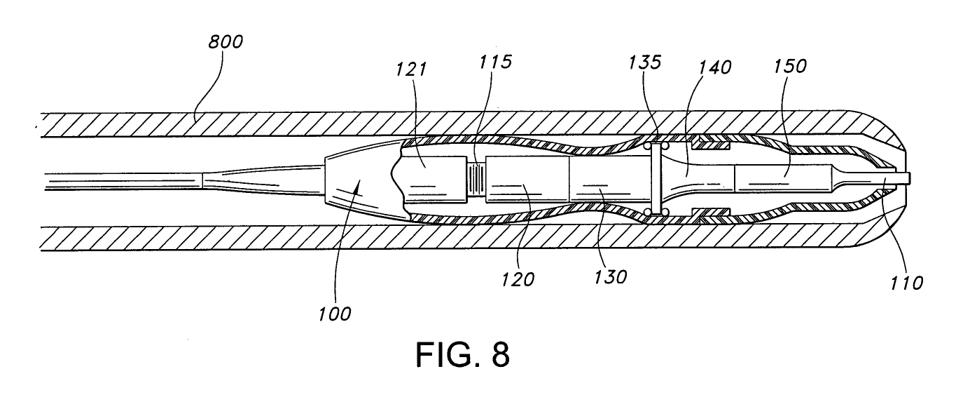

FIG. 8 illustrates still another embodiment where ultrasonic driver 100 is disposed completely within tunneler 800. In this embodiment, head 110 extends to an opening in tunneler 800 sufficient to provide ultrasonic energy at the point of tissue contact as tunneler 800 is advanced through tissue during use.

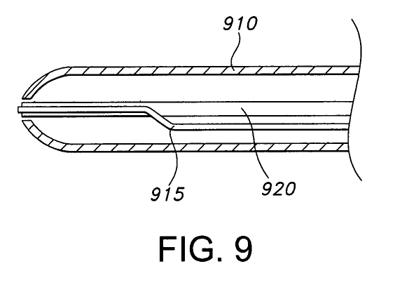

In still another embodiment of the present invention, means for dissecting tissue in the tunneler tip can be provided by direct electrical current instead of ultrasonic energy as described above. In this embodiment, as shown in for example in FIG. 9, a bipolar tip 900 is exposed at the distal end of tunneler 910. The bipolar conductor leads 915 and 920 are coaxial with respect to each other at their distal end region, but at their proximal end are separate. The conductors are separated by a coaxial insulator over that region where they are coaxial (toward the distal end).

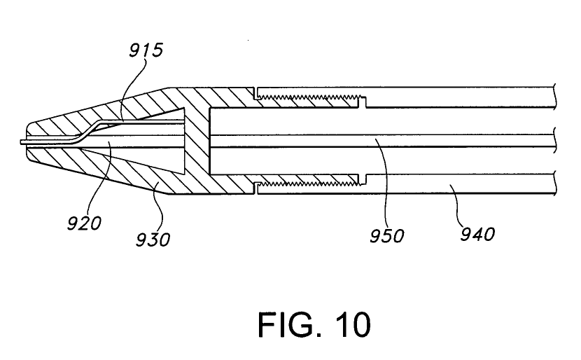

FIG. 10 shows an embodiment having bipolar tunneler tip 930 threadedly connected to tunneler 940. The proximal end of each conductor lead 915 and 920 is in electrical contact with power supply line 950. These bipolar tips are known to those skilled in the art for use in surgical pencils and cauterizing devices. The present invention, however, takes advantage of the delivery of this electrical energy to separate tissue layers during the advancement of the tunneler through the tissue. Also possible for use with the present invention would be a monopolar tip, which configuration would be known by those skilled in the art. With the tissue layer separation as described above, several advantages are realized, including cauterization and trauma reduction. Also as noted above, less force is needed by the operator to advance the tunneler through the tissue.

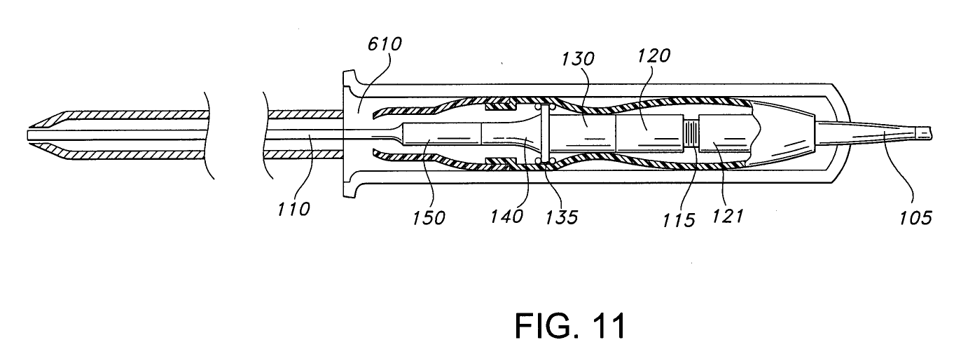

FIG. 11 illustrates still yet another embodiment in which ultrasonic driver 100 is disposed in handle 610 of the device and head 110 extends throughout tunneler 400. The distal tip of the device shown in FIG. 11 does not have a conical tip as shown in the embodiments of FIGS. 2-6, but could have any of those tips disposed on its distal end. As described above, such conical tips could be attached to, or formed as a part of, head 110. Moreover, any combination of the embodiments disclosed above would be understood by one skilled in the art reading this disclosure.

The materials for the tunnelers and tips in accordance with the present invention are typically stainless steel. Other possible materials would be known, however, to those skilled in the ultrasonic and tunneling arts.

Included in the present invention is a method of using the device described above. Specifically, a method in accordance with the present invention includes the steps of advancing a tunneling device into living tissue and separating tissue layers at the tip of the tunneling device as the tunneling device is advanced through the tissue. This method is consistent with the use of the devices described above. The tissue is cauterized in accordance with the delivery of energy, preferably ultrasonic energy or direct electrical energy, as described above. The tunneling procedure generally, however, is that which is known to those skilled in the art. The advantages of the presently disclosed method, however, are described above, and include reduced trauma, reduced recovery time, higher patient comfort, less pain, and ease of use for the person performing the tunneling procedure. These advantages, achieved through this method and using the disclosed device, are a direct result of the delivery of energy (preferably ultrasonic or direct electrical energy) at the distal tip of the tunneler.

Although the invention is illustrated and described herein with reference to specific embodiments, the invention is not intended to be limited to the details shown. Rather, various modifications may be made in the details within the scope and range of equivalents of the claims and without departing from the invention.

What is claimed:

1. A tunneling instrument comprising:
a tubular body having a distal end and a proximal end;
a distal tip disposed at said distal end of said tubular body; and
means for delivering tissue-separating energy to tissue cells proximate said distal tip.

2. The tunneling instrument of claim 1 wherein said tissue-separating energy is ultrasonic energy.

3. The tunneling instrument of claim 1 wherein said tissue-separating energy is electrical energy.

4. The tunneling instrument of claim 1 wherein said means is an ultrasonic driver.

5. The tunneling instrument of claim 3 wherein said electrical energy is provided through one of a monopolar tip or a bipolar tip.

6. The tunneling instrument of claim 1 wherein said distal tip is comprised of a body having a cone-shaped distal end and a proximal end, said tip body comprising an ultrasonic driver which delivers ultrasonic energy to said distal end of said tip body.

7. The tunneling instrument of claim 1 wherein said distal tip is removably attached to said tubular body.

8. The tunneling instrument of claim 1 wherein said distal tip is not removable from said tubular body.

9. The tunneling instrument of claim 1 further comprising a handle disposed proximal to said proximate end of said body, said handle housing an ultrasonic driver to provide said tissue-separating energy to said distal tip.

10. A tunneler tip for use in a surgical tunneling instrument, the tunneler tip comprising:
a body having a cavity disposed therein; and
within said cavity, means for delivering tissue-separating energy to body tissue.

11. The tunneling tip of claim 10 wherein said tissue-separating energy is ultrasonic energy.

12. The tunneling tip of claim 10 wherein said tissue-separating energy is electrical energy.

13. A method of forming a tunnel in living tissue, the method comprising the steps of:
advancing a tunneling device into living tissue; and
separating tissue at the tip of the tunneling device as the tunneling device is advanced through the tissue.

14. The method of claim 13 wherein said separating step includes cauterizing blood vessels within the living tissue.

15. The method of claim 13 wherein said separating is achieved through the delivery of ultrasonic energy to the cells.

16. The method of claim 13 wherein said separating is achieved through the delivery of electrical energy to the cells.
